(19) United States
(12) Patent Application Publication
(10) Pub. No.: US 2007/0288023 A1
(43) Pub. Date: Dec. 13, 2007

(54) SOFT TISSUE REPAIR USING TISSUE AUGMENTS AND BONE ANCHORS

(76) Inventors: Greg Pellegrino, San Diego, CA (US); Joseph C. Tauro, Brick, NJ (US); W. Tate Scott, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/761,783

(22) Filed: Jun. 12, 2007

Related U.S. Application Data
(60) Provisional application No. 60/812,836, filed on Jun. 12, 2006.

(51) Int. Cl. A61B 17/58

(52) U.S. Cl. 606/72

(57) ABSTRACT

Repair and reattachment of damaged soft tissue to bone is facilitated by positioning a biologic or synthetic tissue augment between the soft tissue and the broad footprint of a two-point fixation suture/anchor geometry. Interaction of the tissue augment with the soft tissue is promoted by compression of the augment against the soft tissue over the broad footprint.

### RELATED APPLICATIONS

This application claims the benefit of U.S. Provisional Patent Application No. 60/812,836, filed on Jun. 12, 2006, the disclosure of which is hereby incorporated by reference in its entirety.

### BACKGROUND OF THE INVENTION

1. Field of the Invention

The present invention relates to medical devices and procedures. More particularly, the present invention relates to tissue augments used to support the healing of injured soft tissue.

2. Description of the Related Art

There are several medical procedures where a surgeon needs to attach soft tissue such as tendons or other soft connective tissue to bone. One common example is a torn rotator cuff, where the supraspinatus tendon has separated from the humerus causing pain and loss of ability to elevate and externally rotate the arm. To repair a torn rotator cuff, typically a surgical procedure is used to suture the torn tendon to the bone using a variety of methods. Some procedures utilize large incisions and involve complete detachment of the deltoid muscle from the acromion. Small diameter holes are made in the bone for passing suture material through the bone to secure the tendon. Such large incision procedures are traumatic, causing prolonged pain and recovery time. Other procedures make small incisions and use arthroscopic techniques to attach sutures using either small diameter holes or a bone anchor. Many of these repair procedures involve suturing soft tissue to bone anchors. However, the time required for the tendon to completely heal can be lengthy and the success of the healing variable. Accordingly, there is a need for improved techniques and systems for repairing damaged soft tissue.

### SUMMARY OF THE INVENTION

One embodiment disclosed herein includes a method of attaching soft tissue to bone that involves inserting at least two bone anchors into bone, positioning a tissue augment over the soft tissue, and passing a suture over the tissue augment between the two bone anchors.

Another embodiment disclosed herein includes a kit comprising a tissue augment, suture material, and at least two bone anchors, wherein at least one of the bone anchors is configured to knotlessly fixedly secure the suture material after it is inserted into bone.

Still another embodiment disclosed herein includes a kit comprising suture material, at least two bone anchors, wherein at least one of the bone anchors is configured to knotlessly fixedly secure the suture material after it is inserted into bone, and instructions to a surgeon to position a tissue augment over the soft tissue, insert the two bone anchors, pass the suture material over the tissue augment between the two anchors, and knotlessly fixedly secure the suture material to at least one of the bone anchors.

### BRIEF DESCRIPTION OF THE DRAWINGS

FIGS. 1A-1D are perspective views of various two-point fixation bone anchor/suture geometries.

FIGS. 2A-2H are perspective views of a torn rotator cuff repair procedure using a tissue augment as viewed from a lateral position facing the shoulder.

FIGS. 3A-3G are perspective views of the procedure depicted in FIGS. 2A-2H as viewed from a posterior position facing the shoulder.

### DETAILED DESCRIPTION OF THE CERTAIN EMBODIMENTS

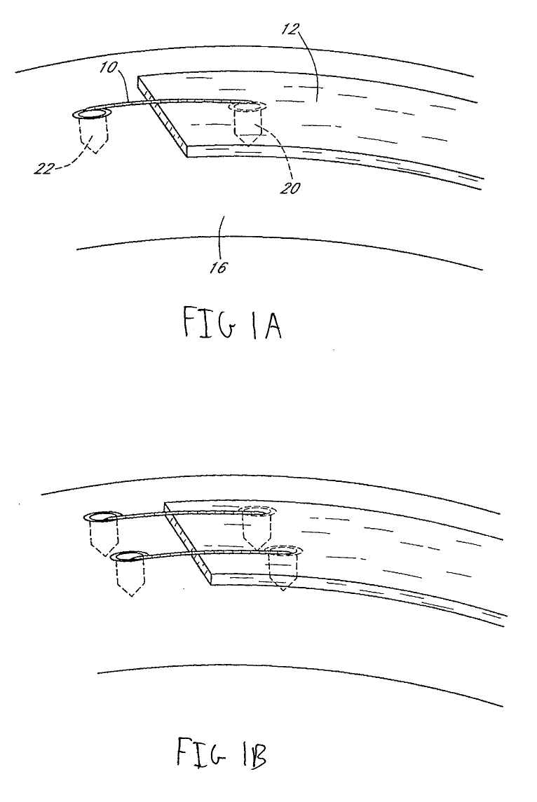

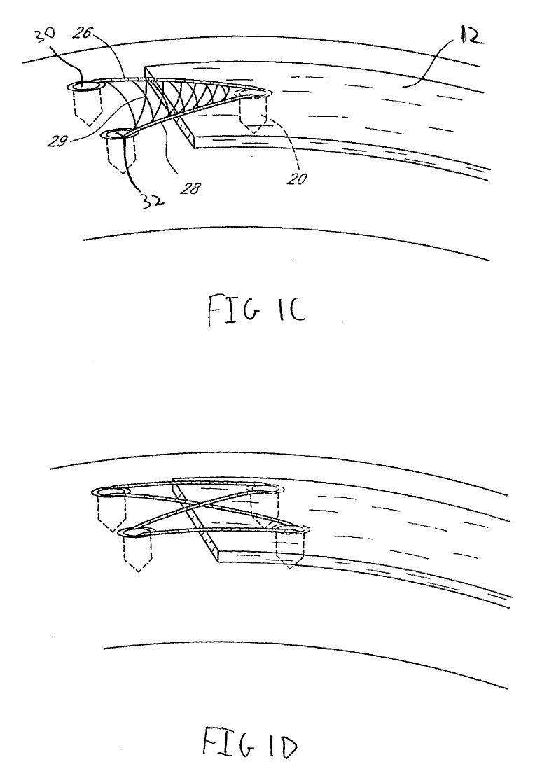

Soft tissue may be re-attached to bone utilizing one or more bone anchors with suture attached thereto. In some embodiments, the suture crosses over the soft tissue between two or more bone anchors. Such an anchor-suture-anchor arrangement may be referred to as “two-point fixation” or “double-row fixation.” This type of arrangement provides for a broad “footprint” of compression of the soft tissue against underlying bone along the entire suture-soft tissue interface. FIGS. 1A-1D are perspective views of four non-limiting two-point fixation arrangements. In FIG. 1A, the soft tissue 12 is compressed against underlying bone 16 by a suture 10 attached between two bone anchors 20 and 22. Bone anchor 20 is positioned beneath the soft tissue 20 and bone anchor 22 is positioned lateral to the soft tissue. It will be appreciated that alternative embodiments include both bone anchors 20 and 22 being located lateral to the soft tissue 12 (e.g., on either side of the soft tissue 12) or both bone anchors 20 and 22 being located underneath the soft tissue 12. FIG. 1B also depicts an arrangement where single strands of suture are stretched between two bone anchors; however, a second anchor-suture-anchor combination is included to increase the compression footprint. FIG. 1C depicts an arrangement where two strands of suture 26 and 28 extend from one bone anchor 20 positioned beneath the soft tissue 12. One strand 26 is attached to a first bone anchor 30 positioned laterally to the soft tissue and the second strand 28 is attached to a second bone anchor 32 also positioned laterally to the soft tissue. In one embodiment, a mesh 29 is optionally attached to the two suture strands 26 and 28 to further increase the compression footprint. FIG. 1D depicts an arrangement where two strands of suture are attached to each bone anchor in a crisscross arrangement.

Those of skill in the art will appreciate that there are any number of anchor-suture-anchor geometries beyond those depicted in FIGS. 1A-1D that may be used to create a compression footprint against the soft tissue. All such arrangements may be used in the methods and systems described herein.

As used herein, “suture” refers to any flexible structure that can be tensioned between two or more anchors and includes, without limitation, traditional suture material, single or multiple stranded threads, or a mesh structure.

In some embodiments, healing of damaged soft tissue and promotion of its reattachment to bone may be aided through the use of a tissue augment. Such an augment may be placed in contact with the soft tissue including the region where the tissue is damaged. In some embodiments, the tissue augment is used to bridge gaps or span a defect between soft tissue including ligaments and tendons as well as gaps between the ligament or tendon to bone insertion points. In some embodiments, the tissue augment is positioned between the soft tissue and suture strands that are part of a two-point fixation anchor-suture-anchor arrangement such as those described above. In these embodiments, the sutures act to compress the tissue augment against the soft tissue over the same broad footprint that the soft tissue is compressed against underlying bone. The two-point fixation geometries promote the interaction of the tissue augment with the soft tissue and thus enhance the tissue healing effects of the tissue augment. Additionally, it is believed that this geometry holds the tissue augment in place during healing and results in a lower incidence of suture pull-out from the augment as compared to methods where the tissue augment is held in place by merely using suture stitches to stitch the augment to the soft tissue or to bone anchors.

The tissue augment may be any biocompatible material that promotes the growth and/or healing of soft tissue. In various embodiments, the augment is a biologic tissue augment or a synthetic tissue augment. The biologic tissue augment may advantageously contain a significant amount of acellular collagen (e.g., at least about 50% of type I collagen) scaffold to promote tissue healing. In some embodiments, the biologic tissue augments are cross-linked. In some embodiments, the biologic tissue augments are obtained from tissue grafts, including allografts and xenografts. Non-limiting tissue grafts include dermal and submucosa tissue grafts.

In one embodiment, a dermal allograft is used, for example from a tissue bank of cadaveric human skin. Such skin may be processed to remove the epidermal and dermal cells resulting in acellular tissue composed of collagen types I, III, IV, VII, elastin, chondroitin sulfate, proteoglycans, and fibroblast growth factor (e.g., such as the commercially available GRAFTJACKET® (Wright Medical Technology, Arlington, Tenn.)). In one embodiment a dermal xenograft is used, for example from porcine or fetal bovine skin. In one embodiment, a single layer of porcine skin is processed to remove hair and epidermis followed by extraction of genetic and cellular components. In one embodiment, the resulting tissue is cross-linked (e.g., using hexamethylenediisocyanate) (e.g., such as the commercially available Zimmer Collagen Repair patch (Tissue Science Laboratories, Covington, Ga.)). In one embodiment, a single layer of fetal bovine skin is processed to remove cells, lipids, and carbohydrates (e.g., such as the commercially available TISSUEMEND® (TEI Biosciences, Boston, Mass.)). In one embodiment, the resulting tissue is cross-linked (e.g., such as the commercially available BIOBLANKET® (Kensey Nash Corp., Exton, Pa.)).

In one embodiment, a submucosa xenograft is used, for example from porcine small intestine submucosa. In one embodiment, the porcine small intestine submucosa is processed to remove noncollagenous components. In one embodiment, the resulting collagenous tissue contains approximately 97% collagen and 2% elastin. In some embodiments, multiple layers of submucosa are laminated followed by cross-linking (e.g., with 1-ethyl-3-(3-dimethylaminopropyl)-carbodiimide) (e.g., such as the commercially available CUFFPATCH™ (Organaogenesis, Canton, Mass.)). In another embodiment, multiple layers of procine small intestine are laminated without subsequent cross-linking (e.g., such as the commercially available RESTORE® graft (Depuy, Warsaw, Ind.)).

In one embodiment, an equine pericardium xenograft is used. In one embodiment, after processing, the equine pericardium comprises approximately 90% of type I collagen and approximately 10% of type II collagen. In one embodiment, the equine pericardium is cross-linked (e.g., such as the commercially available ORTHOADAPT (Pegasus Biologics, Irvine, Calif.)).

Various synthetic tissue augments that may be used include, but are not limited to, polymeric scaffolds containing a matrix or porous architecture that allow for tissue ingrowth into the scaffold. In some embodiments, biologic components that promote tissue healing may be coated onto or infused into the scaffold. Non-limiting synthetic polymers suitable for use as described herein include aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, and blends and co-polymers thereof. In some embodiments, synthetically produced biomolecules are used (e.g., biopolymers such as collagen, elastin, bioabsorbable starches, etc.). Synthetic tissue augments are described in U.S. Application Publication No. 2006/0067967, which is incorporated herein by reference in its entirety.

The tissue augments described herein may be positioned over the soft tissue using either an open, mini-open, or arthoscopic technique. When using an arthroscopic technique, the tissue augment may be folded in such a fashion that it can be inserted through an arthroscopic portal, after which it may be unfolded, positioned, and optionally threaded with suture within the surgical site using graspers or other common arthroscopic surgical instruments.

In some embodiments, the tissue augment is positioned over the soft tissue followed by passing of suture over the augment between two or more bone anchors. In some embodiments, the suture is also threaded through the tissue augment one or more times. Suture may be threaded through the augment such as by using a suture passing needle. Alternatively, pre-formed eyelets are formed in the tissue augment, allowing suture passage using instruments such as a grasper. In one embodiment, the compressive contact between the suture and the augment is sufficient to hold the augment in place without any suture threading.

In one embodiment, a suture strand spanning the tissue augment may be attached at either end to bone anchors after the anchors are inserted and the tissue augment positioned. In such an embodiment, the bone anchors may contain eyelets or other features allowing the suture to be tied to the anchors. In other embodiments, the bone anchors contain a suture capture mechanism that allows the suture to be knotlessly captured after anchor insertion. One such anchor is described in U.S. Application Publication No. 2006-0004364, which is incorporated herein by reference in its entirety. These anchors comprise comprises a proximal member and a distal member configured to clamp the suture material therebetween. In some embodiments, a knotless bone anchor is used that allows coupling of the suture to the anchor prior to anchor insertion followed by fixedly securing the suture after anchor insertion. In some such embodiments, the suture may be tensioned after coupling to the anchor and prior to fixedly securing it to the anchor. For example, bone anchors described in U.S. Application Publication No. 2004-0267317 and U.S. Pat. No. 6,585,730 may be used, both of which are incorporated herein by reference in their entirety.

In one embodiment, the suture strand is attached to one of the bone anchors prior to anchor insertion. The free end of the suture strand may then be passed over the tissue augment and attached to a second bone anchor, either by knot tying or suture capture. The second bone anchor may be inserted either before or after positioning of the tissue augment. In one embodiment, the anchor with pre-attached suture is pierced through soft tissue into the underlying bone, resulting in the suture strand extending through the soft tissue. One such anchor is described in U.S. application Ser. No. 11/557,027, filed Nov. 6, 2006, which is incorporated herein by reference in its entirety. In one such embodiment, the tissue augment is positioned prior to insertion of the anchor with pre-attached suture, allowing the anchor to be pierced through the tissue augment as well as underlying soft tissue. In one embodiment, a surgical grasper having an anchor aperture is used to simultaneously grasp both the soft tissue and the tissue augment in a “sandwich” fashion, thereby ensuring that the tissue augment does not move relative to the soft tissue during anchor insertion. One such surgical grasper is described in U.S. application Ser. No. 11/760,621, filed Jun. 8, 2007, which is incorporated herein by reference in its entirety.

It will be appreciated that any number of bone anchor types may be utilized in the methods described herein. Which combination of bone anchor types are used may depend on the location of the injury in the body (e.g., shoulder, knee, elbow, etc.), the type of soft tissue being repaired (e.g., tendon, ligament, etc.), the type of procedure (e.g., open, arthroscopic, etc.), the severity of the injury, and the skill of the surgeon.

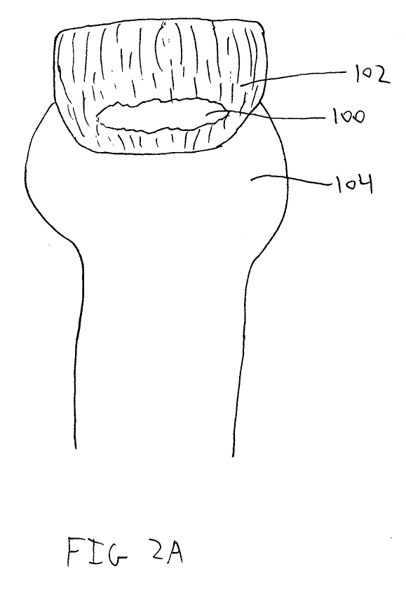

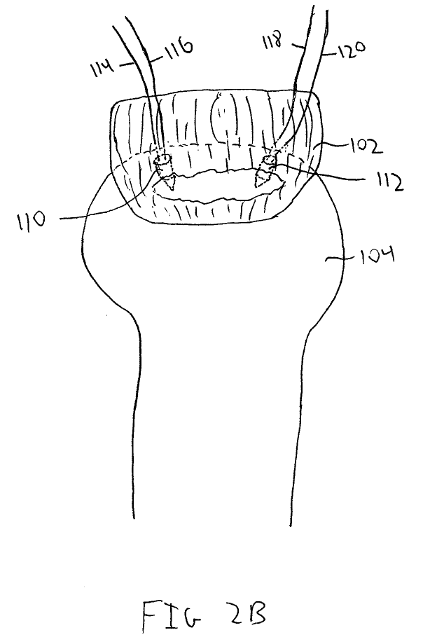

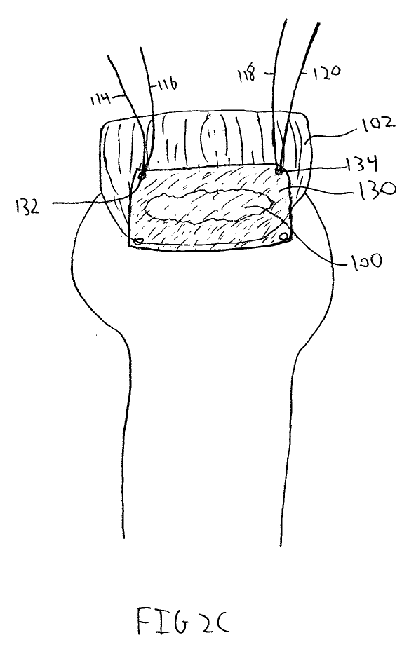

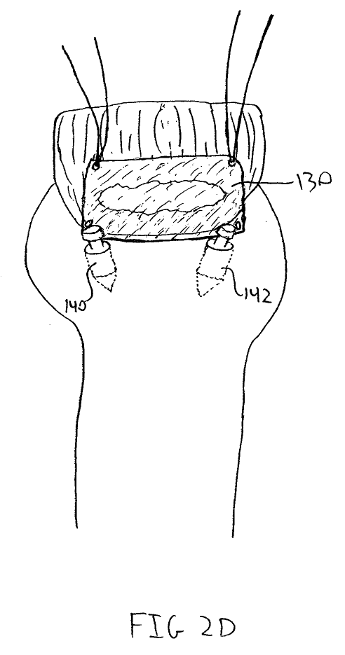

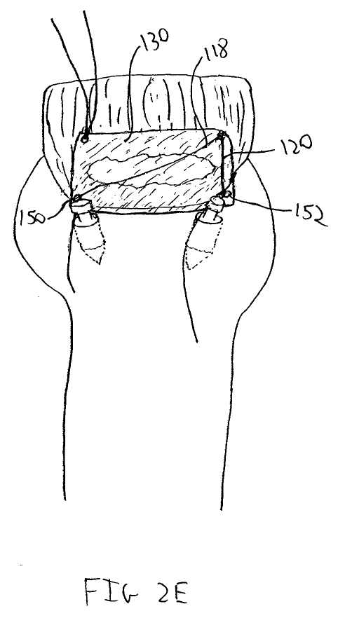

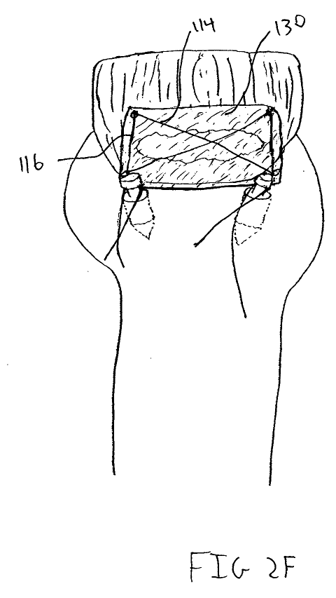

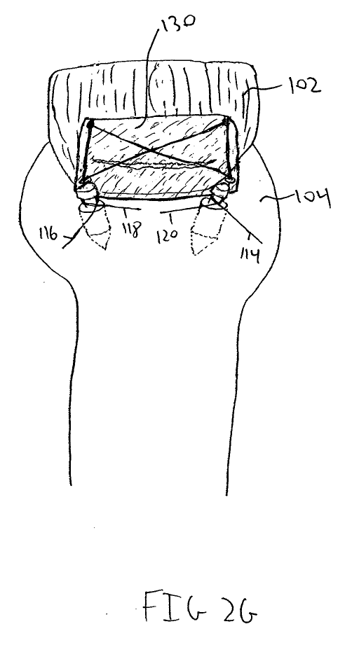

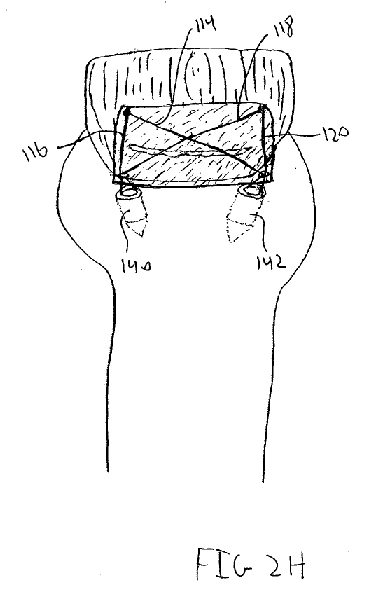

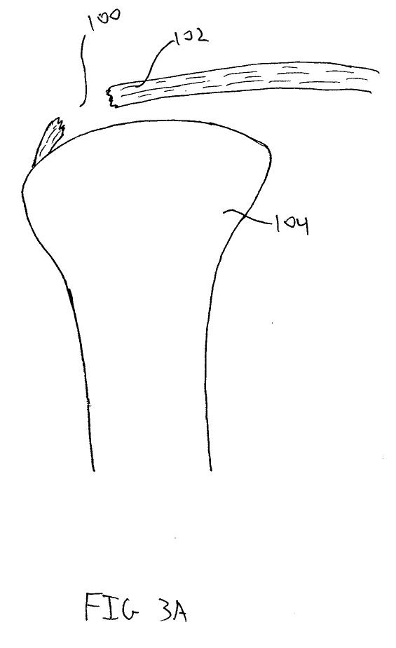

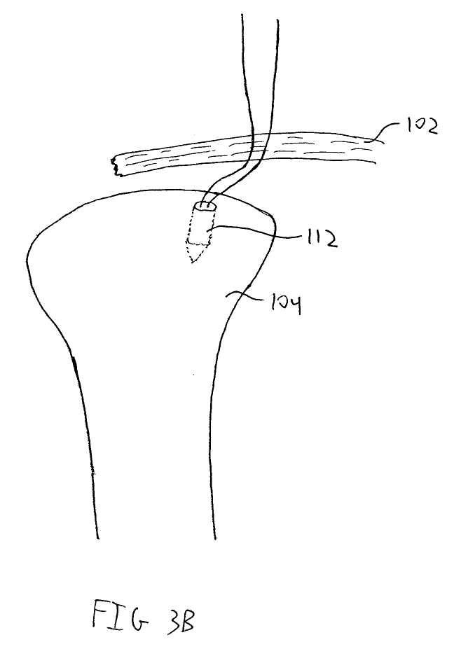

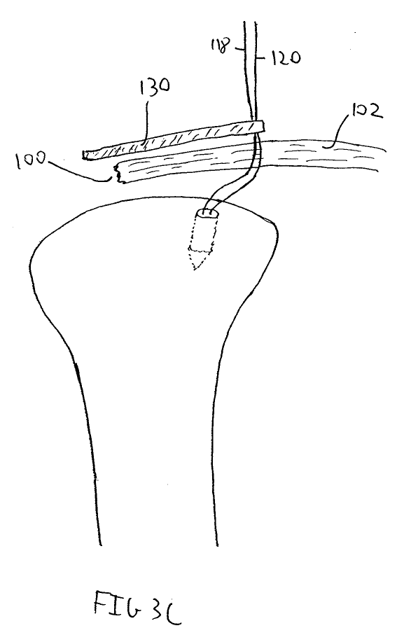

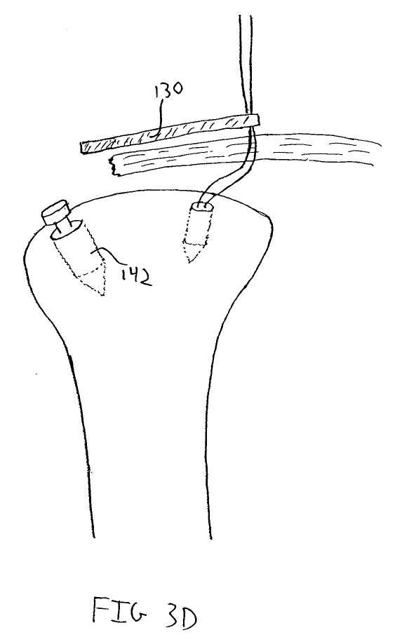

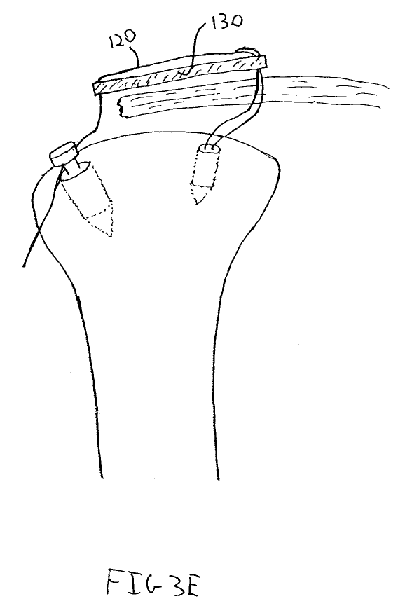

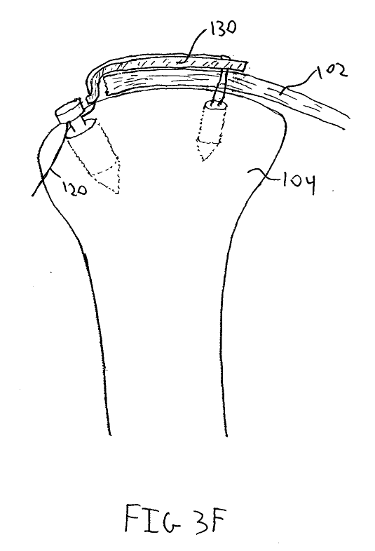

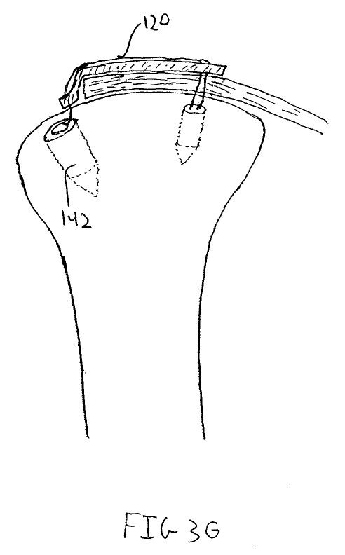

One type of injury well-suited for treatment using the methods described herein is a rotator cuff tear. One non-limiting procedure that may be used to repair a rotator cuff tear is depicted in FIGS. 2A-2H and 3A-3G. FIG. 2A-2H depict the procedure as viewed from a lateral position facing the shoulder. FIG. 3A-3G depict the procedure as viewed from a posterior position facing the shoulder. FIGS. 2A and 3A depict a tear 100 in the rotator cuff 102 near its termination on the humerus 104. As a first step of a repair procedure, a surgeon may prepare the site, such as by debridement of the humerus. Next, as depicted in FIGS. 2B and 3B, two bone anchors 110 and 112, each with two pre-attached sutures, 114, 116, 118, and 120, are pierced through the rotator cuff soft tissue 102 and into the humerus 104 in anterior and posterior medial positions. The bone anchors 110 and 112 may be such that they can be inserted percutaneously and deployed after insertion to prevent pull out, such as the anchors described in U.S. application Ser. No. 11/557,027, filed Nov. 6, 2006, which is incorporated herein by reference in its entirety. The sutures 114, 116, 118, and 120 are left extending from the bone anchors 110 and 112 and through the soft tissue 102.

Next, as depicted in FIGS. 2C and 3C, a tissue augment 130 such as described above is positioned over the soft tissue 102 and tear 100. The sutures 114 and 116 extending from the anterior medial anchor 110 are threaded through the tissue augment 102 near one corner of the augment 130. Similarly, sutures 118 and 120 extending from the posterior medial anchor 112 are threaded through another corner of the augment 130. In one embodiment, apertures such as eyelets 132 and 134 are preformed in the augment 130 to facilitate suture threading. The positioning of the tissue augment 130 and the threading of sutures 114, 116, 118, and 120 may be conducted arthroscopically through a laterally placed portal. In one alternative, the sutures 114, 116, 118, and 120 are threaded through the augment 130 outside of the patient's body and then the treaded augment is inserted and positioned through a laterally placed portal.

As depicted in FIGS. 2D and 3D, after positioning of the tissue augment 130 as described above, two lateral anchors 140 and 142 are inserted lateral to the soft tissue tear 100. In some embodiments, the lateral anchors 140 and 142 are knotless suture capture anchors such as those described in U.S. Application Publication No. 2006-0004364, which is incorporated herein by reference in its entirety. Next, as depicted in FIGS. 2E and 3E, one suture 118 attached to the posterior medial anchor 112 is threaded through the opposite corner of the tissue augment 130. The other suture 120 attached to the posterior medial anchor 112 is threaded through the adjacent lateral corner of the tissue augment 130. Preformed eyelets 150 and 152 may be provided to facilitate suture threading. Similarly, as depicted in FIG. 2F, one suture 114 attached to the anterior medial anchor 110 is threaded through the opposite corner of the tissue augment 130 while the other suture 116 attached to the anterior medial anchor 110 is threaded through the adjacent lateral corner of the tissue augment 130. It can be seen that a crisscross pattern similar to that depicted in FIG. 1D is obtained. It will be appreciated that the suture threading and anchor insertion depicted in FIGS. 2D-2F and 3D-3E may be conducted in any order. In particular, where lateral bone anchors are used that require pre-coupling prior to insertion, the suture threading through the tissue augment will generally be conducted prior to lateral anchor insertion. The threading of sutures 114, 116, 118, and 120 may be conducted arthroscopically through a laterally placed portal. In one alternative, the threading depicted in FIGS. 2C, 2D, 2F, 3C, and 3E may all be conducted outside of the patient's body followed by insertion and positioning of the augment 130 through a laterally placed portal.

Next, as depicted in FIGS. 2G and 3F, the sutures 114, 116, 118, and 120 are tensioned to reduce the torn rotator cuff 102 down onto the humerus 104 and to compress the tissue augment 130 down onto the rotator cuff 102. Finally, as depicted in FIGS. 2H and 3G, the lateral bone anchors 140 and 142 are actuated to capture the sutures 114, 116, 118, and 120, fixedly securing them in a tensioned state. The tensioning of sutures 114, 116, 118, and 120 may be conducted arthroscopically through a laterally placed portal. In one alternative embodiment, one lateral bone anchor 140 (or 142) is inserted through a laterally placed portal followed by tensioning and capture of sutures 116 and 118 (or 114 and 120) prior to insertion of the other lateral bone anchor 142 (or 140) and tensioning and capture of sutures 114 and 120 (or 116 and 118). In this manner, a single laterally placed portal may be used to conduct all suture passing, tissue augment positioning, lateral anchor insertion, suture tensioning, and suture capture.

It will be appreciated that many variations of anchor/suture geometry, anchor type, and order of steps may be utilized to achieve the result of a tissue augment and rotator cuff being compressed downward over a broad footprint. In one variation of the procedure described in FIGS. 2A-2H and 3A-3G, after medial anchor insertion (i.e., anchors 110 and 112 in FIGS. 2B and 3B), the sutures 114, 116, 118, and 120 are pulled out through a laterally placed portal. One strand of suture from each anchor (e.g., sutures 116 and 120) are then threaded along the side of the augment 130 by passing each suture up through a medial eyelet 132 or 134 and then down through a lateral eyelet 150 or 152. This threading is conducted outside of the patient's body. After this step, the sutures 116 and 120 will be positioned along the sides of the augment as depicted in FIGS. 2F and 2E, respectively. The remaining suture strands 114 and 118 are then threaded up through medial eyelets 132 and 134, respectively, and then passed back through the lateral portal and up through a medially placed portal. The sutures 114 and 118 may then be pulled by the surgeon causing the augment 130 to be pulled down through the lateral portal and into position over the rotator cuff. During this procedure, the surgeon may hold sutures 116 and 120 taut to provide “rails” along which the augment 130 may slide as sutures 114 and 118 are pulled. Finally, sutures 116 and 120 are threaded through lateral eyelets 150 and 152 to create the crisscross pattern depicted in FIG. 2F. Lateral anchors 140 and 142 (see FIG. 2D) may then be inserted and deployed through the same lateral portal through which the augment 130 was pulled.

One embodiment includes a kit provided to a surgeon that comprises the necessary bone anchors, suture material, and tissue augments as described above. In some embodiments, required and optional surgical instruments are also included in the kit, including graspers, suture setters, probes, and anchor insertion tools.

Although the invention has been described with reference to embodiments and examples, it should be understood that numerous and various modifications can be made without departing from the spirit of the invention. Accordingly, the invention is limited only by the following claims.

What is claimed is:

1. A method of attaching soft tissue to bone, comprising:
inserting at least two bone anchors into bone;
positioning a tissue augment over the soft tissue; and
passing a suture over the tissue augment between the two bone anchors.

2. The method of claim 1, wherein the soft tissue is a rotator cuff.

3. The method of claim 1, wherein at least one of the anchors is inserted through the soft tissue into underlying bone.

4. The method of claim 1, wherein at least one of the anchors is inserted through the tissue augment and the soft tissue into underlying bone.

5. The method of claim 1, wherein the tissue augment is positioned over the soft tissue after inserting at least one of the two anchors.

6. The method of claim 1, wherein the tissue augment is positioned over the soft tissue prior to inserting at least one of two anchors.

7. The method of claim 1, wherein the suture is attached to at least one of the two bone anchors after the tissue augment is positioned over the soft tissue and the suture is passed over the tissue augment.

8. The method of claim 7, wherein the suture is coupled to the at least one anchor outside of a patient's body.

9. The method of claim 8, wherein the suture is fixedly secured to the at least one anchor after the at least one anchor is inserted into bone.

10. The method of claim 7, wherein the at least one anchor is inserted into bone without any suture coupled thereto.

11. The method of claim 7, wherein the suture is knotlessly attached to the at least one anchor.

12. The method of claim 1, wherein the suture is attached to at least one of the bone anchors prior to insertion.

13. The method of claim 1, comprising threading the suture at least once through the tissue augment.

14. The method of claim 13, wherein threading the suture through the tissue augment comprises threading the suture through a preformed aperture in the tissue augment.

15. The method of claim 13, wherein the suture is threaded through the tissue augment outside of a patient's body.

16. The method of claim 1, comprising:
inserting first and second anchors into bone;
positioning the tissue augment over the soft tissue;
inserting third and fourth anchors into bone;
attaching a first suture strand between the first and third anchors over the tissue augment;
attaching a second suture strand between the first and fourth anchors over the tissue augment;
attaching a third suture strand between the second and third anchors over the tissue augment; and
attaching a fourth suture strand between the second and fourth anchors over the tissue augment.

17. The method of claim 16, comprising tensioning the first, second, third, and fourth suture strands.

18. The method of claim 17, wherein the third and fourth anchors are inserted and the first, second, third, and fourth suture strands tensioned through the same arthroscopic portal.

19. The method of claim 1, wherein the tissue augment is positioned over the soft tissue using a surgical grasper.

20. The method of claim 19, wherein the soft tissue and the tissue augment are simultaneously grasped by the surgical grasper.

21. The method of claim 1, comprising:
inserting first and second anchors into bone, wherein the first and second anchors comprise suture strands extending therefrom;
threading at least one suture strand through the tissue augment outside of a patient's body; and
pulling on the at least one suture strand, thereby pulling the tissue augment into the patient's body and positioning it over the soft tissue.

22. The method of claim 1, wherein the tissue augment comprises a biologic tissue augment.

23. The method of claim 1, wherein the tissue augment comprises a synthetic tissue augment.

24. A kit comprising:
a tissue augment;
suture material; and
at least two bone anchors, wherein at least one of the bone anchors is configured to knotlessly fixedly secure the suture material after it is inserted into bone.

25. The kit of claim 24, wherein the suture material is pre-attached to at least one of the bone anchors.

26. The kit of claim 24, wherein the bone anchor that is configured to fixedly secure the suture material after it is inserted into bone comprises a proximal member and a distal member configured to clamp the suture material therebetween.

27. The kit of claim 24, wherein the tissue augment comprises a biologic tissue augment.

28. The kit of claim 27, wherein the biologic tissue augment comprises at least about 50% of type I collagen.

29. The kit of claim 28, wherein the collagen is cross-linked.

30. The kit of claim 27, wherein the biologic tissue augment is derived from a tissue allograft.

31. The kit of claim 30, wherein the tissue allograft is a dermal allograft.

32. The kit of claim 27, wherein the biologic tissue augment is derived from a tissue xenograft.

33. The kit of claim 32, wherein the tissue xenograft is a dermal xenograft.

34. The kit of claim 33, wherein the dermal xenograft is porcine skin.

35. The kit of claim 33, wherein the dermal xenograft is fetal bovine skin.

36. The kit of claim 32, wherein the tissue xenograft is a submucosa xenograft.

37. The method of claim 36, wherein the submucosa xenograft is porcine small intestine submucosa.

38. The kit of claim 32, wherein the tissue xenograft is equine pericardium.

39. The kit of claim 24, wherein the tissue augment comprises a synthetic tissue augment.

40. The kit of claim 39, wherein the synthetic tissue augment comprises a synthetic polymer.

41. The kit of claim 40, wherein the synthetic polymer has a matrix or porous architecture.

42. A kit comprising:
suture material;
at least two bone anchors, wherein at least one of the bone anchors is configured to knotlessly fixedly secure the suture material after it is inserted into bone; and
instructions to a surgeon to position a tissue augment over the soft tissue, insert the two bone anchors, pass the suture material over the tissue augment between the two anchors, and knotlessly fixedly secure the suture material to at least one of the bone anchors.
